(19) United States
(12) Patent Application Publication
(10) Pub. No.: US 2015/0153368 A1
(43) Pub. Date: Jun. 4, 2015

(54) Early Predictive Markers of Pre-Eclampsia

(71) Applicant: Universite Laval, Quebec (CA)

(72) Inventors: Jean-Francois Bilodeau, Quebec (CA); Pierre Julien, Quebec (CA)

(21) Appl. No.: 14/541,299

(22) Filed: Nov. 14, 2014

Related U.S. Application Data
(63) Continuation-in-part of application No. PCT/CA2013/000490, filed on May 16, 2013.
(60) Provisional application No. 61/648,151, filed on May 17, 2012.

(51) Int. Cl. G01N 33/88; G01N 33/92

(52) U.S. Cl. CPC G01N 33/88; G01N 33/92; G01N 2800/368; G01N 2800/50

(57) ABSTRACT

The present invention relates to a method for measuring levels of class VI isoprostane, an early predictive marker of pre-eclampsia (PE). The present invention also relates to an assay and diagnostic kit for performing the method of predicting PE by measuring the level of this marker and optionally establishing a ratio over other markers such as polyunstaturated fatty acids (PUFA) or beta-carotene. In particular, the method determines the level a class VI isoprostane in a blood sample from a pregnant woman prior to the appearance of symptoms of PE.

### CROSS REFERENCES TO RELATED APPLICATIONS

This application is a continuation-in-part of PCT Application No. PCT/CA2013/000490, filed May 16, 2013 which claims priority to U.S. Provisional Application Ser. No. 61/648,151, filed May 17, 2012, entitled “Early Predictive Markers of Pre-Eclampsia”, the entire contents of which are incorporated by reference herewith.

### FIELD OF THE INVENTION

The present invention relates to a method and assay for predicting pre-eclampsia (PE). The present invention also relates to a kit for performing the assay of predicting PE.

### BACKGROUND OF THE INVENTION

PE affects approximately 3-5% of all pregnancies and is a leading cause of maternal death in North America and the UK. This disease, or the threat of onset, is the commonest cause of elective premature delivery, accounting for approximately 15% of all premature births. PE is defined according to the guidelines of the International Society for the Study of Hypertension in Pregnancy and includes amongst other factors: gestational hypertension and proteinuria. Gestational hypertension is defined as two recordings of diastolic blood pressure of 90 mm Hg or higher at least 4 h apart, and severe pressure of 110 mm Hg or higher at least 4 h apart or one recording of diastolic blood pressure of at least 120 mm Hg. Proteinuria is defined as excretion of 300 mg or more in 24 h or two readings of 2+ or higher on dipstick analysis of midstream or catheter urine specimens if no 24 h collection was available.

Women are classified as previously normotensive or with chronic hypertension before 20 weeks' gestation. For previously normotensive women, PE is defined as gestational hypertension with proteinuria and severe PE as severe gestational hypertension with proteinuria. For women with chronic hypertension, superimposed PE is defined by the new development of proteinuria. The measurement of blood pressure and testing for proteinuria in all pregnant women is carried out predominantly for the detection of PE. These procedures and the care of affected women and of the premature children make considerable demands on healthcare resources.

There is no widely accepted or accurate method for the early prediction of PE. Elevation of the blood pressure and detection of protein in the urine occur when the disease process is well established. Detection of an abnormality of the blood flow to the uterine artery by Doppler ultrasound in women who later develop PE has been of some predictive use but this abnormality has been found to be relatively non-specific and for this reason has not been adopted in routine clinical practice.

Although some plasma/urine biochemical markers have been shown to be abnormal in the disease process, no single marker has proven to be of adequate sensitivity for use as a predictive indicator. For example the use of placenta growth factor (PlGF) alone as a predictive indicator of PE has been proposed, but the predictive power of this marker could not be determined with any certainty. For example, International patent application WO 98/28006 suggests detecting PlGF alone or in combination with vascular endothelial growth factor (VEGF) in order to predict the development of PE.

U.S. Pat. No. 5,891,622 teaches that isoprostanes are used to quantify an oxidative stress associated to numerous pathologies. This patent is focused on the use of ELISAs to measure free, conjugated or esterified isoprostanes (IsoPs) at large in plasma, urine, cerebrospinal, bile and joint fluids. Alternatively, GC/MS can be used for the determination of IsoPs.

U.S. Pat. No. 7,833,795 describes a method to assess cardiovascular risk using isoprostanes and liquid chromatography/tandem mass spectrometry in urine and plasma exclusively. Although, it is true that PE increases the risk of being affected by cardiovascular diseases later in life, PE is not a cardiovascular disease per se. The focus of the patent is on three isomers: 8,12-iso-iPF_{2α}-VI, 8-iso-PGF_{2α}, and iPF_{2α}-VI. According to the authors, additional parameters are required to predict cardiovascular risk and comprise: thromboxane metabolite and a PGI₂ metabolite in urine, blood pressure, blood level of C-reactive protein, blood level of interleukin-6 (IL-6), blood level of soluble intracellular adhesion molecule-1 (sICAM-1), blood level of monocyte chemoattractant protein-1 (MCP-1), blood level of homocysteine, presence or extent of atherosclerotic plaques, and presence of one or more genetic predispositions for elevated cardiovascular risk.

Although, there is no widely used treatment for PE (other than premature delivery), Chappell et al. [1] have shown a significant reduction in PE in high risk women given supplements of vitamin C and vitamin E. In this study, risk was assessed by a test of relatively low sensitivity. More accurate and robust identification of women at risk would target those women most likely to benefit from this, or alternative, prophylactic therapies. Those identified at lower risk could be provided with less intensive and less expensive antenatal care.

Despite previous encouraging results of antioxidant vitamin trials, vitamin C and E supplementation did not reduce the rate of PE, but increased the risk of fetal loss or perinatal death and preterm pre-labor rupture of membranes in a large Canadian cohort [2]. However, other antioxidants need to be investigated.

Therefore, there remains a need for accurate and early identification of women at risk of developing and suffering from PE, such that treatments can be elaborated.

### SUMMARY OF THE INVENTION

It has now been found that the blood lipid profile, particularly, the fatty acid profile, optionally in combination with a specific marker to provide a ratio, provides the much needed predictive parameters for the desired early prediction and/or diagnosis of PE.

The present invention provides a method of specific prediction of PE in a subject, comprising determining in a maternal biological sample a level of a class VI isoprostane, wherein said amount of class VI isoprostane above a control is indicative that said subject is at risk of developing PE.

In accordance with an aspect of the invention, there is provided a method for measuring blood isoprostane profile in a pregnant woman at risk of developing preeclampsia (PE), comprising the steps of: a) extracting lipids from a said pregnant woman's biological sample; b) measuring total level of F₂-isoprostane class VI; c) optionally, measuring total level of 15(R)-PGF_{2α} or fatty acids from said sample; d) optionally, establishing a ratio of F₂-isoprostane class VI over blood fatty acids for said pregnant woman; and e) comparing said amount or said ratio with a control level or ratio from a control population or individual representative of said pregnant woman; and f) optionally, reporting said comparison from step e) to said subject's treating physician; wherein when said level or ratio is at least about 10% higher than said control level or ratio, said physician may diagnose pre-eclampsia and, optionally take measures to monitor or treat said pregnant woman.

The present invention provides a method of specific prediction of PE in a pregnant woman, comprising determining in a maternal sample a level of isoprostane 5-iPF_{2α}-VI and/or iPF_{2α}-VI, wherein said amount of 5-iPF_{2α}-VI and/or iPF_{2α}-VI above a control level is indicative that said woman is at risk of developing PE.

The present invention provides a method of specific prediction of PE in a pregnant woman, comprising determining in a maternal sample the ratio of total isoprostanes over 15(R)-PGF_{2α} or over blood fatty acids, wherein a higher ratio is indicative that said pregnant woman is at risk of developing PE.

The present invention provides a method of specific prediction of PE in a pregnant woman, comprising determining in a maternal sample the ratio of total isoprostanes over polyunsaturated fatty acids (PUFA), wherein a higher ratio is indicative that said pregnant woman is at risk of developing PE.

The present invention provides a method of specific prediction of PE in a pregnant woman, comprising determining in a maternal sample the ratio of class VI isoprostanes over omega-3 and/or omega-6 polyunsaturated fatty acids (PUFA), wherein a higher ratio is indicative that said pregnant woman is at risk of developing PE.

The present invention provides a method of specific prediction of PE in a pregnant woman, comprising determining in a maternal sample the ratio of isoprostane 5-iPF_{2α}-VI and/or iPF_{2α}-VI over 15(R)-PGF_{2α} or over arachidonic acid, wherein a higher ratio is indicative that said pregnant woman is at risk of developing PE.

The present invention provides a method of specific prediction of PE in a pregnant woman, comprising determining in a maternal sample the ratio of isoprostane 5-iPF_{2α}-VI and/or iPF_{2α}-VI over the ratio of omega-3 to omega-6 polyunsaturated fatty acids (PUFA), wherein a higher ratio is indicative that said pregnant woman is at risk of developing PE.

The present invention provides, a method for predicting the appearance of PE in a subject comprising the steps of: a) determining in a sample the level of total isoprostane; b) determining in said sample the level of 15(R)-PGF2α or blood fatty acid profile; c) establishing the ratio of total isoprostane over 15(R)-PGF2α or over blood fatty acid profile; wherein said ratio above a control ratio is indicative that said pregnant woman is at risk of developing PE.

Alternatively, the present invention also provides an assay for predicting the appearance of preeclampsia (PE) in a pregnant woman comprising the steps of: a) obtaining a sample from said pregnant woman; b) assessing amount of total isoprostane in said sample; c) assessing blood fatty acid profile in said sample; d) establishing a ratio of total isoprostanes over blood fatty acid profile for said woman; e) comparing said ratio with a control level for a population or an individual representative of said pregnant woman; and f) determining if said comparing of step e) is higher than about 10% of said control level; and g) optionally reporting said determination from step f) to said subject's treating physician.

Optionally, the method also comprises the additional step of taking measures to place this woman under surveillance or tight monitoring, and/or adjusting anti-oxidant intake.

It has been found that by measuring the markers or ratios mentioned above, it is possible to determine with high specificity and sensitivity whether a subject is likely to develop PE.

Other features and advantages of the invention will be apparent from the following detailed description, the drawings, and the claims.

### DETAILED DESCRIPTION OF THE INVENTION

### Brief Description of the Drawings

Having thus generally described the aspects of the invention, reference will now be made to the accompanying drawings, showing by way of illustration, particular embodiments thereof, and in which the figures represent:

FIG. 1: HPLC gradient used for the analysis of F₂-isoprostanes. Solvent A: H₂O+0.01% acetic acid; solvent B: ACN+0.01% acetic acid; Solvent C: MeOH+0.01% acetic acid.

FIG. 2: Representation of the structures of F₂-isoprostanes used to setup the HPLC-MS-MS method.

FIG. 3: Representation of the structure of the deuterated F₂-isoprostane internal standards used to setup the HPLC-MS-MS method.

FIG. 4A and FIG. 4B: Chromatograms of class III F₂-isoprostanes obtained by HPLC-MS-MS. FIG. 4A: Two ng/ml of each analyte monitored at transition 353.3/193.3 m/z. Letters (A-L) beside peaks in panel correspond to analytes represented in FIG. 2. FIG. 4B: Two ng/ml of each internal standard were monitored at transition 357.3/197.2 m/z. Letters (P,Q) beside peaks in panel correspond to standards represented in FIG. 3.

FIG. 5A and FIG. 5B: Chromatograms of class IV F₂-isoprostanes obtained by HPLC-MS-MS. FIG. 5A: Two ng/ml for each analyte were monitored at transition 353.0/127.0 m/z. Letter (L) in panel corresponds to an analyte represented in FIG. 2. FIG. 5B: Two ng/ml for each internal standard were monitored at transition 357.0/127.0 m/z. Letter (R) refers to a standard represented in FIG. 3.

FIG. 6A and FIG. 6B: Chromatograms of class VI F₂-isoprostanes obtained by HPLC-MS-MS. FIG. 6A: Two ng/ml for each analyte were monitored at transition 353.0/115.0 m/z. Letters (M-O) in panel corresponds to analytes represented in FIG. 2. FIG. 6B: Two ng/ml for each internal standard were monitored at transition 364.6/115.0 m/z. Letters (S-U) refer to standards represented in FIG. 3.

FIG. 7A and FIG. 7B. Comparison between conventional HPLC-MS/MS (FIG. 7A) and three-dimensional separation of isoprostanes of class VI using the combination of HPLC, ion mobility and tandem mass spectrometry (MS/MS) (FIG. 7B). Plasma sample was spiked with 0.1 ng of isoprostane standards. Peak identification: A: iPF_{2α}-VI; B: 5-iPF_{2α}-VI; C: 5-8,12-iso-iPF_{2α}-VI.

FIG. 8. Receiver operating characteristic (ROC) curves for isoprostane (iPF_{2α}-VI+5 iPF_{2α}-VI) normalized to plasma volume ( [symbol] ), ω-3/ω-6 ratio ( [symbol] ) and to UI ω-3/β-carotene ( [symbol] ). Omega-3 fatty acids (ω-3)=C18:3 ω3+C18:4 ω3+C20:3 ω3+C20:4 ω3+C20:5 ω3+C22:5 ω3+C22:6 ω3. Omega-6 fatty acids (/ω-6)=C18:1ω6+C18:2 ω6+C18:3 ω6+C20:2n6+C20:3 ω6+C20:4 ω6+C22:2 ω6+C22:4 ω6+C22:5 ω5. Unsaturation index for omega-3 (UI ω-3)=(% Monoenoic×1)+(% Dienoic×2)+(% Trienoic×3)+(% Tetraenoic×4)+(% Pentaenoic×5)+(% Hexaenoic×6) of ω-3 fatty acids.

### DEFINITIONS AND ABBREVIATIONS

With respect to the invention presented herein, the following definitions and abbreviations are used, wherein:

The abbreviation “AA” means arachidonic acid.

The abbreviation “iP” means isoprostane, whereas the abbreviation iPF_{2α} means F_{2α} isoprostane.

The abbreviation “PUFA” means polyunsaturated fatty acids.

The abbreviation “15(R)-PGF_{2α}” means the C-15 epimer of prostaglandin F_{2α}.

The term “(±)5-iPF₂ₐ₋VI” means the mixture of iPF2α-VI and 5-iPF2α-VI.

The term “pre-eclampsia” (PE) as used herein is defined according to the guidelines of the International Society for the Study of Hypertension in Pregnancy, as described above.

The “maternal sample” is taken from a pregnant woman and can be any sample from which it is possible to measure the markers mentioned herein. Preferably the sample is blood. The samples can be taken at any time from about 10 weeks gestation. Preferably the sample is taken at between 12 and 24 weeks gestation, more preferably the samples are taken before 20 weeks.

The term “sensitivity” is defined as the proportion of true positives (i.e. will develop PE) identified as positives in the method.

The term “specificity” is defined as the proportion of true negatives (i.e. will not develop PE) identified as negatives in the method.

The term “specific prediction of pre-eclampsia” as used herein means that the method of the present invention is used to specifically predict the development of PE. In particular, the method of the present invention enables one to determine whether an individual is likely to develop PE.

The abbreviation “UI” means unsaturation index and is calculated in the following manner: unsaturation index for omega-3 (UI ω-3)=(% Monoenoic×1)+(% Dienoic×2)+(% Trienoic×3)+(% Tetraenoic×4)+(% Pentaenoic×5)+(% Hexaenoic×6) of fatty acids; or unsaturation index for omega-6 (UI ω-6)=(% Monoenoic×1)+(% Dienoic×2)+(% Trienoic×3)+(% Tetraenoic×4)+(% Pentaenoic×5)+(% Hexaenoic×6) of fatty acids.

### DETAILED DESCRIPTION OF PARTICULAR EMBODIMENTS

Applicant has obtained samples of blood from pregnant women who were considered at risk of PE on the basis of the uterine artery Doppler test or because they had had the disease in a previous pregnancy. Blood samples were obtained respectively twice from 12 to 18 weeks and 24 to 26 weeks of pregnancy. A selection of biochemical markers implicated in PE were measured, including vitamin C, homocysteine, plasma lipids and 8-epi prostaglandin F_{2α} but none proved to be effective in prediction. We found that the ratio of total isoprostanes over blood fatty acids increased prior to the onset of the disease. Combinations of these markers proved to be excellent in the sensitive and specific prediction of subsequent PE.

### Method

The present invention therefore provides, a method of specific prediction of PE in a subject comprising the steps of:
a) determining in a maternal sample the level of total isoprostanes;
b) determining in said maternal sample the level of blood lipids;
c) establishing a ratio of total isoprostanes over blood lipids;
   wherein said ratio above (or under) a pre-determined ratio is indicative that said pregnant woman is at risk (or not, respectively) of developing PE.

More particularly, in step a) the method comprises the determination of the level of iPF_{2α}-VI and/or 5-iPF_{2α}-VI.

Still, more particularly, in step b) the method comprises the determination of blood fatty acids.

Alternatively, in step c), the method comprises the determination of the ratio between 5-iPF_{2α}-VI and/or iPF_{2α}-VI over arachidonic acid (AA).

Still, alternatively, in step b) the method comprises the determination of omega-3 PUFA and omega-6 PUFA, and establishing a ratio of omega-3 PUFA over omega-6 PUFA herein defined as ω3/ω6 ratio.

In a particular aspect of the invention, step c) comprises the determination of the ratio 5-iPF_{2α}-VI and/or iPF_{2α}-VI over ω3/ω6 ratio.

In a particular aspect of the invention, step c) comprises the determination of the ratio 5-iPF_{2α}-VI and/or iPF_{2α}-VI over 15(R)-PGF_{2α}.

In a particular aspect, there is provided the method as defined herein further comprising before step a), a step of separating isoprostanes from said extracted lipids, wherein said separating is carried out by mass spectrometry.

In an alternative aspect, there is provided the method as defined herein wherein said F₂-isoprostane class VI is selected from the group consisting of: 5-iPF_{2α}-VI and iPF_{2α}-VI.

In an alternative aspect, there is provided the method as defined herein, wherein said fatty acid is selected from the group consisting of: arachidonic acid, omega-3 and omega-6 polyunsaturated fatty acids (PUFA).

In an alternative aspect, there is provided the method as defined herein, wherein said ratio is a ratio of (±)iPF_{2α}-VI isoprostane over the ratio of omega-3 to omega-6 polyunsaturated fatty acids (PUFA), whereby when said ratio is above a control ratio is indicative that said pregnant woman is at risk of developing PE.

In an alternative aspect, there is provided the method as defined herein, wherein said ratio is a ratio of (±)iPF_{2α}-VI isoprostane over percentage of omega-3 polyunsaturated fatty acids (PUFA) in said sample, whereby when said ratio is above a control ratio is indicative that said pregnant woman is at risk of developing PE.

Alternatively, there is provided the method as defined herein, wherein said ratio is a ratio of (±)iPF_{2α}-VI isoprostane over a ratio of omega-3 PUFA unsaturation index over concentration of beta-carotene, whereby when said ratio is above a control ratio is indicative that said pregnant woman is at risk of developing PE.

In an alternative aspect, there is provided the method as defined herein, wherein said ratio is a ratio of (±)iPF_{2α}-VI over a ratio of omega-3 PUFA unsaturation index over concentration of beta-carotene over CoQ10, whereby when said ratio is above a control ratio is indicative that said pregnant woman is at risk of developing PE.

In an alternative aspect, there is provided the method as defined herein, wherein said ratio is a ratio of (±)iPF_{2α}-VI over a ratio of omega-3 PUFA unsaturation index over concentration of beta-carotene over CoQ10 over alpha-tocopherol, whereby when said ratio is above a control ratio is indicative that said pregnant woman is at risk of developing PE.

The method the present invention may be performed in conjunction with other tests for diagnostic indicators, such as blood pressure, level of uric acid etc.

### Ratio and Control Level

In order to determine whether the level or ratio of the markers referred to above is greater than normal, the normal level (i.e. control) or ratio of the relevant control population or individual needs to be determined. The relevant control population or individual may be defined based on, for example, ethnic background or any other characteristic that may affect normal levels of the markers. The relevant population or individual for establishing the normal level or ratio of the markers is preferably selected on the basis of low risk for PE (i.e. no known risk marker for PE, such as previous PE, diabetes, prior hypertension etc.).

Particularly, the control population or individual is selected from the group consisting of: an individual in a normal population devoid of PE symptom, a non-pregnant woman, said pregnant subject prior to pregnancy, and same pregnant subject prior to 10 week of pregnancy.

Once the normal levels are known, the measured levels can be compared and the significance of the difference determined using standard statistical methods. If there is a statistically significant difference between the measured level and the normal level, then there is a significant risk that the individual from whom the levels have been measured will develop PE.

Particularly, there is a significant difference when the sample level is increased by at least about 10% compared to the control level, particularly at least about 15%, more particularly at least about 20%.

Of course, the present invention teaches that a certain ratio when higher is indicative of preeclamptic condition. Should a person skilled in the art decide to inverse the ratio taught (such as for example 15(R)-PGF_{2α} over class-VI iP), then a decreased ratio will lead to the same conclusion (see Table 3). The method and assay as claimed also encompass this reverse ratio.

It can be seen that the level of sensitivity and specificity can be altered by altering the control level. In some situations, e.g. when screening large numbers of women at low risk of PE, it is important to have high specificity. In other situations, it may be important to have a balance between high sensitivity and specificity, e.g. when considering individual women at high risk of PE a balance between high sensitivity and specificity is needed.

### Assay

The present invention therefore provides, an assay for predicting the appearance of PE in a subject comprising the steps of:
a) obtaining a biological sample from said subject;
b) assessing the amount of total isoprostanes in said sample;
c) assessing blood fatty acid profile in said sample;
d) establishing a ratio of total isoprostane over blood fatty acids for said subject;
e) comparing said ratio with control ratio for a population representing said subject;
f) determining if said comparing of step e) is above said control level; and
g) reporting said determination from step f) to said subject's treating physician.

### Kit

The present invention also provides a diagnostic kit for performing the method of the present invention. The kit comprises reagents required to determine the level of the markers being measured. Suitable agents for assaying for the markers include enzyme linked immunoassay reagents, RIA reagents and reagents for Western blotting.

A further aspect of the present invention relates to a kit for performing MS (in particular MS/MS) for quantifying class-VI isoprostanes in a subject's biological sample, this kit comprising: a standard for 5-iPF_{2α}-VI and/or iPF_{2α}-VI for calibration and validation; instructions for calibrating and validating said MS/MS, and instructions for measuring said class-VI isoprostanes. Particularly, the standards are deuterated. More particularly the kit may also comprise standards for fatty acids ω-3 and/or ω-6 (such as arachidonic acid: AA) and/or phospholipids containing fatty acids ω-3 and/or ω-6.

### Subject

Particularly, the subject is a pregnant woman. The sample can be taken at any time from about 10 week gestation. Particularly, the sample can be taken at any time prior to the 24th week of pregnancy. More particularly, the sample is taken at between 12 and 20 weeks gestation.

### Sample

The maternal sample can be any sample from which it is possible to measure the markers mentioned above. Particularly, the sample is selected from: blood, red or white blood cell membranes, plasma, serum, urine, cerebrospinal fluid, bile or joint fluid. More particularly, the sample is taken from blood, plasma, serum or blood cell membranes. Most particularly, the sample is plasma or serum. More particularly, the markers are measured from blood cell membranes contained in the sample.

### Use as Marker

More than twenty biochemical markers have been shown previously to be associated with established PE and there would be no logical prior reason for choosing 5-iPF_{2α}-VI and/or iPF_{2α}-VI in any prospective longitudinal study for assessment of use as predictive indicators. Moreover very few groups have evaluated any individual marker prospectively in the same women from whom samples were taken at intervals throughout their pregnancy. Importantly none has measured the different markers in the same women, unlike in the present application.

According to another aspect of the invention, there is provided the use of F2-isoprostanes class-VI such as isoprostane 5-iPF_{2α}-VI and/or iPF_{2α}-VI and/or the ratio of F2-isoprostanes class-VI over blood fatty acids; and/or the ratio of F2-isoprostanes class-VI over arachidonic acid or over ω-3/ω-6 or over 15(R)-PGF_{2α} as a predictive marker(s) for pre-eclampsia in a pregnant woman, particularly, prior to 20^{th} week gestation, more particularly prior to the appearance of first symptoms.

### Methodologies for Measuring the Markers

In accordance with another aspect of the present invention, there is provided a method for measuring blood isoprostane profile in a pregnant woman at risk of developing preeclampsia (PE), comprising the steps of:
a) extracting lipids from a said pregnant woman's biological sample;
b) performing mass spectrometry on said extracted lipids to separate isoprostanes and measuring total level of F₂-isoprostane class VI;
c) optionally, measuring total level of 15(R)-PGF_{2α} or fatty acids from said sample;
d) optionally, establishing a ratio of F2-isoprostane class VI over 15R-PGF_{2α} or over blood fatty acid profile for said subject;
e) comparing said amount or said ratio with a control level or ratio from a control population or individual representing said subject;
f) reporting said comparison from step e) to said subject's treating physician;
wherein when said level or ratio is at least about 15% higher than said control level or ratio, said physician may diagnose pre-eclampsia and, optionally take measures to monitor or treat the subject.

Particularly, the total fatty acid profile can be determined by gas chromatography GC-FID (flame ionization detection) or GC-MS (mass spectrometry) or any other means well known in the art.

More particularly, the mass spectrometry technology used in step b) is: ion mobility MS.

Particularly, the levels of class VI isoprostanes can be assessed by one, two or more steps of mass spectrometry (MS-MS), particularly when preceded by liquid chromatography or by an ionization source such as for example: HPLC-MSMS, HPLC-MS-MS-MS; MALDI (Matrix-assisted laser desorption/ionization)-MS-MS, MALDI-MS-MS-MS, GC-MS-MS or ELISA or any other means well known in the art.

More particularly, the levels of polyunsaturated fatty acids (PUFA) can be assessed by GC-FID (flame ionization detection), GC-MS or GC-MS-MS or any other means well known in the art.

### Immunoassay

Alternatively, the assay can take the form of an enzyme linked immunoassay (ELISA) or a radio-immuno assay (RIA).

### Therapeutic Intervention

Particularly, the invention also comprises the additional step of taking measures to place the woman having an increased risk of PE under surveillance or tight monitoring for avoiding life threatening events for the foetus. Alternatively, the woman can be prescribed anti-oxidant therapy and monitored for further symptoms to develop or stabilize.

### Therapeutic Target

Of course, an alternative aspect of the invention is to provide a marker useful for developing therapeutic strategies to avoid, prevent or treat PE.

The marker of the present invention may also be used in order to monitor the efficiency of a prophylactic treatment for preventing the development of PE, wherein a reduction in the risk of developing PE will be indicative of the efficacy of the prophylactic treatment.

The present invention offers many benefits. In addition to facilitating accurate targeting of interventions e.g. vitamin supplements or antioxidants, considerable saving on health care resources can be potentially gained due to stratification of antenatal care and reduced neonatal special care costs. In the research and development area, identification of high risk patients will greatly facilitate future clinical trials. At present due to inadequate methods of prediction, large numbers of pregnant women unnecessarily receive interventions in clinical trials.

The following examples are intended to illustrate, rather than limit, the invention.

### EXAMPLES

### Example 1

### Materials and Methods

Measurement of F₂-isoprostanes by HPLC-MS-MS

### Materials

All F₂-isoprostanes and prostaglandin isomers, including 8-iso-15(R)-PGF_{2α}, Ent-8-iso-15(S)-PGF_{2α}, 8-iso-PGF_{2α}, Ent-8-iso-PGF_{2α}, 8-iso-PGF_{2β}, 11β-PGF_{2α}, 15(R)-PGF_{2α}, 5-trans-PGF_{2α}, PGF_{2α}, Ent-PGF_{2α}, PGF_{2β}, iPF_{2α}-IV, (±)5-iPF_{2α}-VI, (±)8,12-iso-iPF_{2α}-VI were purchased from Cayman Chemical (Ann Arbor, Mich., USA) as well as deuterated standards 8-iso-PGF_{2α}-d4, PGF_{2α}-d4, iPF_{2α}-IV-d4, iPF_{2α}-VI-d4, (±) 5-iPF_{2α}-VI-d11, and (±)8,12-iso-iPF_{2α}-VI-d11. Butylated hydroxytoluene (BHT) was bought from Sigma-Aldrich (Oakville, ON, Canada) and sodium chloride (ACS grade) was obtained from Laboratoire Mat (Québec, QC, Canada). All other reagents and solvents were HPLC grade and were purchased from VWR International Inc. (Ville Mont-Royal, QC, Canada).

### Preparation of Solutions

A solution called internal standard containing 50 ng/mL of each deuterated analyte (8-iso-PGF_{2α}-d4, PGF_{2α}-d4, iPF_{2α}-IV-d4, iPF_{2α}-VI-d4, (±)5-iPF_{2α}-VI-d11, and (±)8,12-iso-iPF_{2α}-VI-d11) was prepared in 0.01% acetic acid. A stock solution containing 1 μg/mL of each compound (8-iso-15(R)-PGF_{2α}, 8-iso-PGF_{2α}, 15(R)-PGF_{2α}, 5-trans-PGF_{2α}, PGF_{2α}, iPF_{2α}-IV, (±)5-iPF_{2α}-VI and (±)8,12-iso-iPF_{2α}-VI) was also prepared in 0.01% acetic acid. The previous solutions were used to prepare two sets of working solutions in which concentration ranged from 2 ng/mL to 80 ng/mL in 0.01% acetic acid. First set of working solution was diluted to obtain standard curves for each analyte (10 μL of working solution, 10 μL of internal standard, 80 μL of water containing 10% (v/v) acetonitrile and 0.01% (v/v) acetic acid). The second set of working solutions was diluted to obtain quality controls.

### Sample Preparation

### Erythrocyte Cell Membrane Extraction [3]

Ten μL of a BHT solution (1% in ethanol) was added to 250 μL of freshly thawed whole blood and the volume was completed to 1 ml with water. Samples were mixed, incubated for 5 min. at room temperature, and were centrifuged for 15 min. at 21 000×g. The supernatant was discarded and 1 mL of a sodium chloride solution (0.9% (w/v) in water) was added. Samples were remixed and centrifuged for 12 minutes at 21 000×g. The previous steps were done twice in order to wash correctly erythrocyte cell membranes. Finally, supernatant was discarded and 250 μL of water was added to each tube. Aliquots were stored at −20° C. until extraction of isoprostanes.

Extraction of Isoprostanes from Plasma

Isopostanes were extracted from plasma using an adapted version of the method developed by Taylor [4]. Ten μL of a BHT solution (1% in ethanol) and 10 μL of the internal standard were added to 250 μL of freshly thawed plasma. Then, the samples were diluted with 250 μL of water and mixed with 500 μL of an hydrolysis solution (1 mL 50% (w/w) KOH, 1 mL water, 10 mL methanol). The resulting mixture was incubated at 37° C. for 60 minutes. One hundred μL of formic acid 0.05% (v/v) and 90 μL of hydrochloric acid 5 N were added to each tube to stop the reaction. Samples were mixed and extracted twice with 1.5 mL of hexane. The organic phase was discarded. The aqueous phase was then extracted three times with 1.5 mL of 3:1 ethyl acetate:hexane. The organic phase was collected and combined in polypropylene conical tubes. Finally, extracts were evaporated to dryness under a stream of dry nitrogen and reconstituted with 100 μL of water containing 10% (v/v) acetonitrile and 0.01% (v/v) acetic acid.

Extraction of Isoprostanes from Whole Blood

Isoprostanes were extracted from whole blood as described above for the plasma but 150 μL of blood was used instead. The samples were diluted to 350 μL with water. Only one extraction with hexane is performed though. After final reconstitution, the extract was filtered by a nanosep MF GHP 0.45 μM at 13 000 RPM for 1 min. (Pall Life Science) before injection to the HPLC.

Extraction of Isoprostanes from Erythrocyte Cell Membrane

Isoprostanes were extracted from erythrocyte cell membranes as described above from plasma but the totality of aliquots obtained after erythrocyte cell membranes extraction was used. No BHT solution was added in this case.

### Chromatography

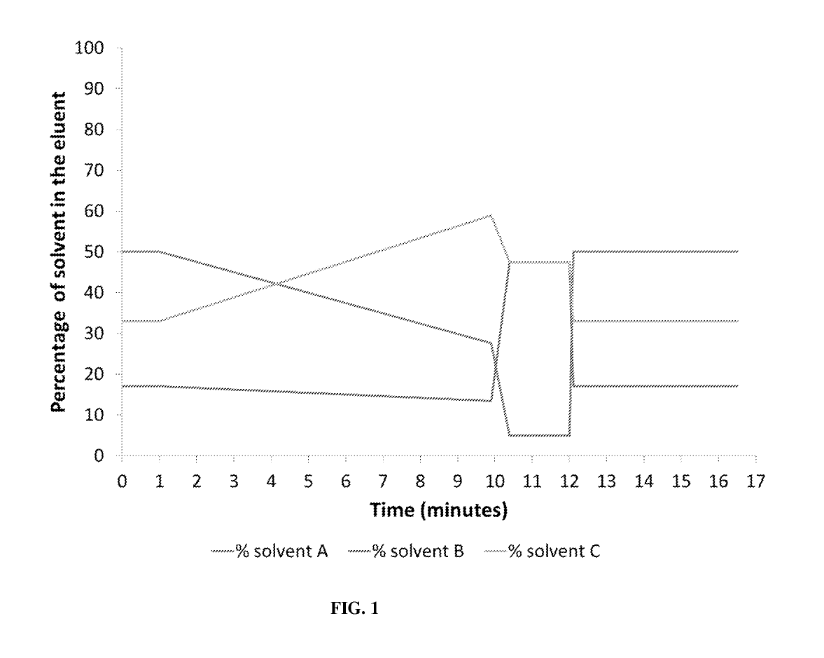

The chromatography was carried out using a Shimadzu Prominence system (Columbia, Md., USA). A Kinetex XB-C18 100 Å column (100×3.0 mm, 2.6 μm) was used preceded by a 4.0×2.0 mm C18 SecurityGuard Cartridges. Both were from Phenomenex (Torrance, Calif., USA). The column oven temperature was controlled at 30° C. and the isoprostanes separation was performed using a gradient of three solvents at a flow rate of 0.45 mL/min (see FIG. 1). Solvent A was composed of 0.01% (v/v) acetic acid in water, solvent B consisted of 0.01% (v/v) acetic acid in acetonitrile and solvent C was composed of 0.01% (v/v) acetic acid in methanol. First, solvent B was held at 17% for 1 min, while solvent C was held at 33% followed by a linear gradient over 8.9 min to 13.5% B and 58.9% C. Then, a linear gradient over 0.5 min to 47.5% B and 47.5% C were programmed. The latter conditions were maintained for 1.6 min and were decreased to 17% B and 33% C in 0.1 min respectively. The final condition were held for 4.4 min to complete the 16.5 min run. The injection volume was 40 μL for samples, quality controls and the standard curve.

### Mass Spectrometry

The HPLC was coupled to a 3200 QTRAP LC/MS/MS system from AB Sciex (Concord, ON, Canada) through a Turbo V ion source using the electrospray ionization probe according to the method described in Larose et al. [8]. The mass spectrometer was operated in negative mode. Curtain gas (CUR), collision gas (CAD), ion source gas 1 (GS1) and ion source gas 2 (GS2) were respectively set at 37, 7, 45 and 55. Ionspray voltage (IS) was set at −4100 V and source temperature was set at 700° C. Class III F₂-isoprostanes and their internal standard, 8-iso-PGF_{2α}-d4 and PGF_{2α}-d4 (class III-d4), were monitored in the multiple-reaction monitoring (MRM) mode using the transitions 353.3/193.2 and 357.3/197.2 respectively. Class IV F₂-isoprostanes and their internal standard, iPF_{2α}-IV-d4 (class IV-d4), were monitored using the transitions 353.3/127.0 and 357.0/127.0. Finally, class VI isoprostane and their internal standard, (±)5-iPF_{2α}-VI-d11, and (±)8,12-iso-iPF_{2α}-VI-d11 (class VI-d11), were analysed using the transitions 353.0/115.0 and 364.6/115.0 respectively. Table 1 summarizes analyte-specific mass spectrometry parameters for each transition. Quantification was performed using Analyst 1.4.2 Software.

TABLE 1
Multiple Reactions monitoring (MRM) transitions and analyte-
specific mass spectrometry parameters.

| Class of F2- | MRM transitions | DP¹ | EP² | CE³ | CEP⁴ | CXP⁵ |
|---|---|---|---|---|---|---|
| isoprostanes | (m/z) | (V) | (V) | (V) | (V) | (V) |
| Class III | 353.3 → 193.2 | −50.0 | −7.0 | −34.0 | −20.0 | −4.0 |
| Class III-d4 | 357.3 → 197.2 | −50.0 | −7.0 | −34.0 | −20.0 | −4.0 |
| Class IV | 353.0 → 127.0 | −45.0 | −7.0 | −33.0 | −17.0 | −2.0 |
| Class IV-d4 | 357.0 → 127.0 | −45.0 | −7.0 | −33.0 | −17.0 | −2.0 |
| Class VI | 353.0 → 115.0 | −47.0 | −7.0 | −30.0 | −21.0 | −2.0 |
| Class VI-d4 | 357.0 → 115.0 | −47.0 | −7.0 | −30.0 | −21.0 | −2.0 |
| Class VI-d11 | 364.6 → 115.0 | −47.0 | −7.0 | −30.0 | −21.0 | −2.0 |

¹Declustering potential.
²Entrance potential.
³Collision energy.
⁴Collision cell entrance potential.
⁵Collision cell exit potential.

### Method Validation

The lower limit of quantification (LLOQ) was defined as the concentration to which the S/N ratio was equal to 10 with a precision below 20% and an accuracy of ±20% of the nominal concentration. Determination of intra-day precision was done by analysing a pool of plasma samples from three non-pregnant women (Innovative Research, Novi, Mich., USA) spiked with 10 μL of working solutions containing either 0 ng/mL, 7 ng/mL and 20 ng/mL of each analyte (n=4 per concentration). This experiment was done on three consecutive days in order to evaluate inter-day precision (n=12 per concentration). Concentration of each F₂-isoP was determined in a pooled plasma sample and accuracy was determined for the samples spiked with the 7 and 20 ng/mL solutions. The recovery was evaluated by comparing signal obtained for plasma spiked before extraction with 10 μL of solutions containing 7 ng/mL, 10 ng/mL and 20 ng/mL of each analyte with signal obtained for plasma spiked after extraction with the corresponding working solutions. Matrix effects were evaluated by post column infusion at 10 μL/min of a solution containing 100 ng/mL of each following molecules: 8-iso-PGF_{2α}, 8-iso-PGF_{2α}-d4, iPF_{2α}-IV, iPF_{2α}-IV-d4, 5-iPF_{2α}-VI, 5-iPF_{2α}-VI-d11. During post column infusion, an extract of plasma was injected concomitantly using the described HPLC-MS/MS method above.

### Fatty Acid Profile

The fatty acid composition of the plasma and erythrocyte membranes were performed according to the method previously described [3, 5]. The fatty acids from plasma were isolated according to a method previously described [6]. Briefly, a solution of chloroform:methanol (2:1, by volume) was used to extract lipids from plasma. Then, phospholipids were separated by thin layer chromatography using a mix of isopropyl ether:acetic acid (96:4) as elutant and fatty acids were methylated following a trans esterification reaction using a mix of methanol:benzene (4:1) and acetyl chloride. Methylated fatty acids were finally analyzed by gas chromatography coupled with a flame ionization detector (GC-FID) as explained elsewhere [7].

### Example 2

### Results

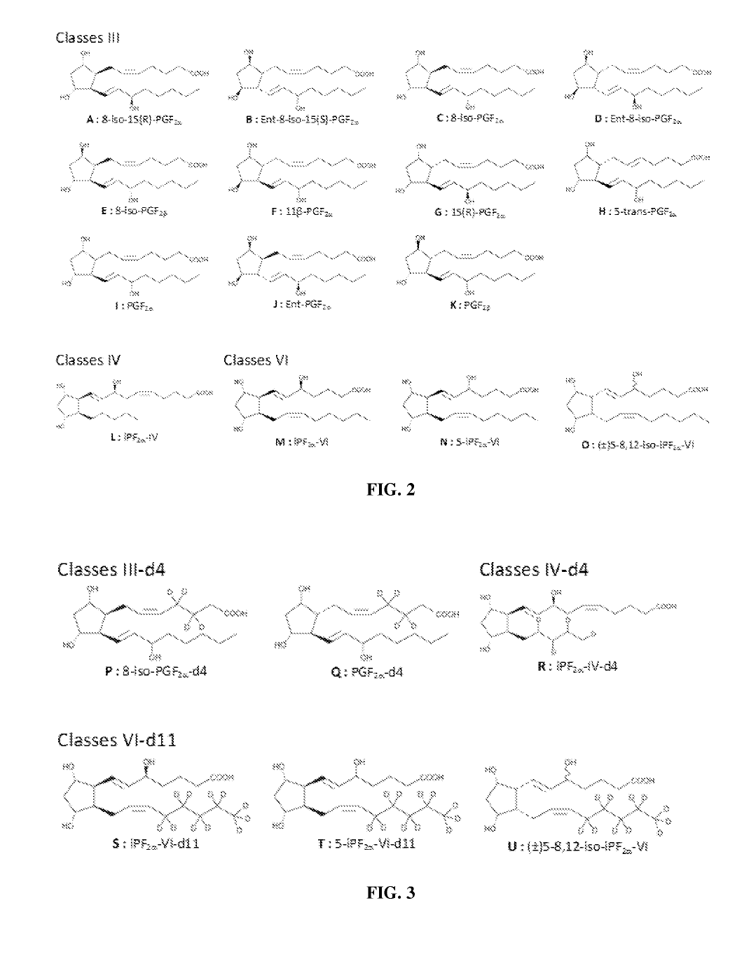

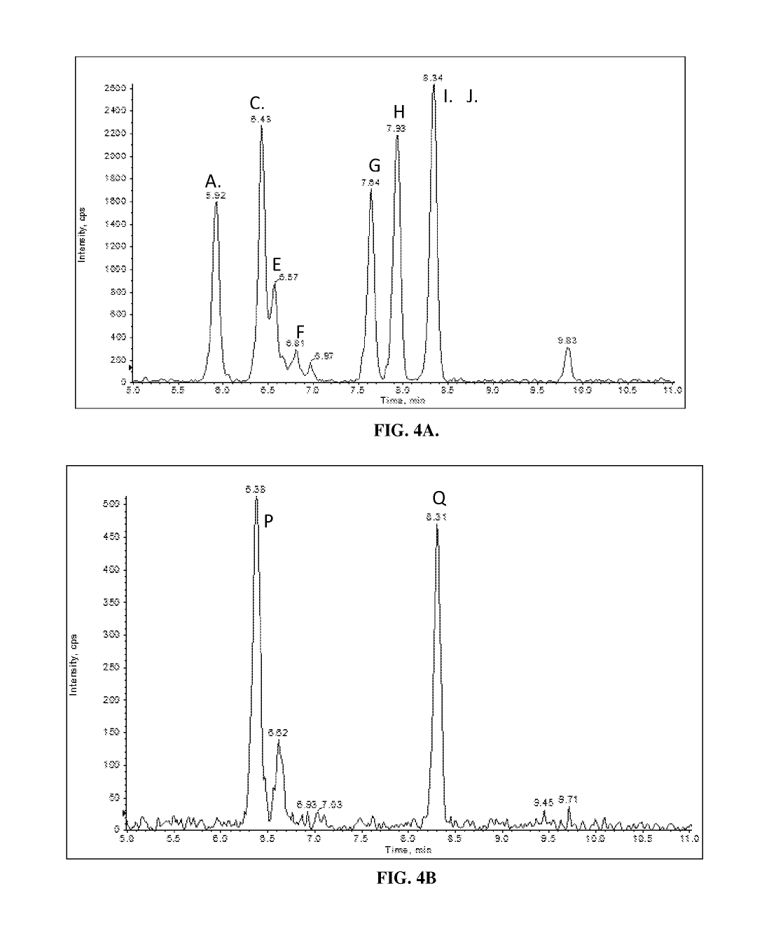

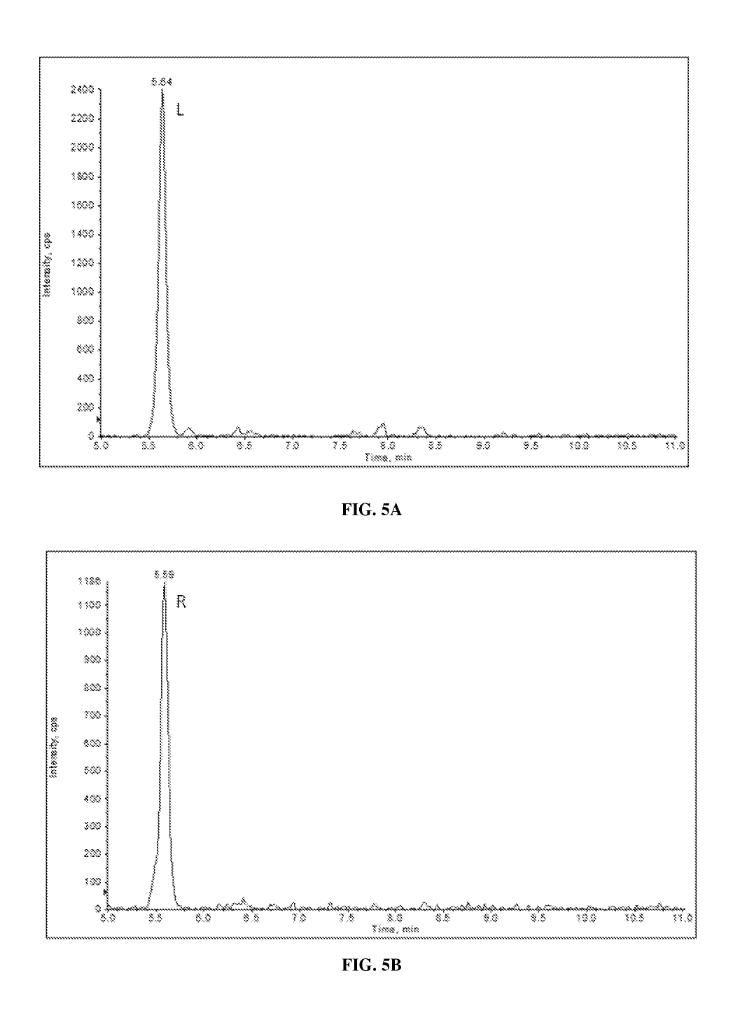

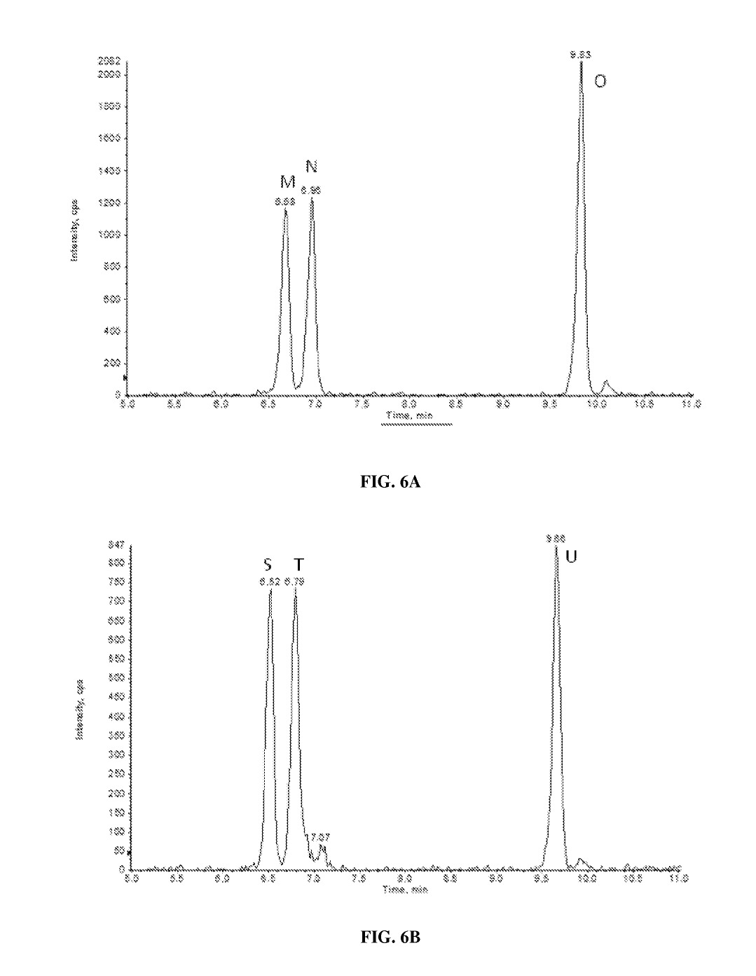

The detailed structures of commercially available F₂-isoprostanes used to develop the described HPLC-MS-MS method in Example 1 are shown in FIG. 2. Deuterated standards shown in FIG. 3 were used to identify and control for the yield of the isoprostanes extraction or other potential biases throughout the whole experimental procedure. Typical chromatograms for F₂-isoprostanes of class III, IV and VI obtained from HPLC-MS-MS analysis are showed respectively in FIGS. 4 to 6. The letters in chromatograms correspond to the structures detailed in FIGS. 2 and 3. For class III F₂-isoprostanes, it was not always possible to separate all isomers distinctly. Isomers A and B co-eluted in the same peak on the chromatogram of FIG. 4A. The same phenomenon occurred for isomer C and D and finally I, J and K. The latter indicates that the measurements of commonly studied 8-iso-(15R)-PGF_{2α} and 8-iso-PGF_{2α} are possibly inaccurate because of the co-elution as shown here (FIG. 4A). Moreover, ELISA kit displayed cross-reactivity for several of these isomers. On the other hand, one class IV isomer can be clearly detected using the only available commercial standard (FIG. 5). It was possible to distinctly separate by chromatography three isomers of class VI respectively the iPF_{2α}-VI, 5-iPF_{2α}-VI, and the (±)8,12-iso-iPF_{2α}-VI.

TABLE 2
Correlations between F₂-isoprostanes of class VI (iPF_{2α}-
VI + 5-iPF_{2α}-VI) and the fatty acid profile in the
maternal plasma of 12-18 weeks preeclamptic pregnancies.

Preeclampsia

| | r | P | N |
|---|---|---|---|
| Total Omega-6 | 0.625 | 0.00718* | 17 |
| Total Saturated | 0.446 | 0.0707 | 17 |
| fatty acids | | | |

*P < 0.05.

The correlations observed in Table 2 led us to investigate the ratio between F₂-isoprostanes and the plasmatic fatty acid profile. The ratio of class VI F₂-isoprostanes with the omega-3/omega-6 ratio further improved the significant difference observed between control and preeclamptic pregnancies (Table 3). Of note, ratio of F₂-isoprostanes can also be used to predict PE.

In Table 3, the F₂-isoprostanes of class VI are predictive of preeclampsia in the first half of the pregnancy since the levels of iPF_{2α}-VI+5-iPF_{2α}-VI is 21% higher in preeclamptic than control pregnancies. Also, we observed several correlations between class VI F₂-isoprostanes and the fatty acid profile as shown in Table 2. Interestingly, iPF_{2α}-VI+5-iPF_{2α}-VI correlated exclusively with omega-6 and saturated fatty acids in preeclampsia and not in controls (Table 2). In contrast, class VI F₂-isoprostanes specifically correlated with trans fatty acids mostly in control pregnancies.

TABLE 3
F₂-isoprostanes of class VI (iPF_{2α}-VI + 5-iPF_{2α}-VI)
differed from controls in the first half of pregnancy with
preeclampsia (PE).
Ratio to isoprostanes of class III and fatty acids either
normalize the data or further increase the significance.

| | | | (omega-3/ |
|---|---|---|---|
| | | | omega-6)/ |
| | iPF₂ -VI + | 15R-PGF₂/ | (iPF₂ -VI + |
| | 5-iPF₂ -VI | (iPF₂ -VI + 5-iPF₂ -VI) | 5-iPF₂ -VI) |
| Controls | 155 ± 11 | 0.839 ± 0.048 | 1.262 ± 0.094 |
| pg/ml plasma | | | |
| PE | 188 ± 13 | 0.651 ± 0.078 | 0.802 ± 0.086 |
| pg/ml plasma | | | |
| P (T-test) | 0.018*¹ | 0.0411* | 0.003**¹ |

Data are means ± SEM.
¹Mann-Whitney Rank Sum Test (non-parametric).

### Example 3

### Improved Separation of Isoprostanes

We have recently improved the separation of isoprostanes of class VI using a newly developed ion mobility technique. Ion mobility mass spectrometry

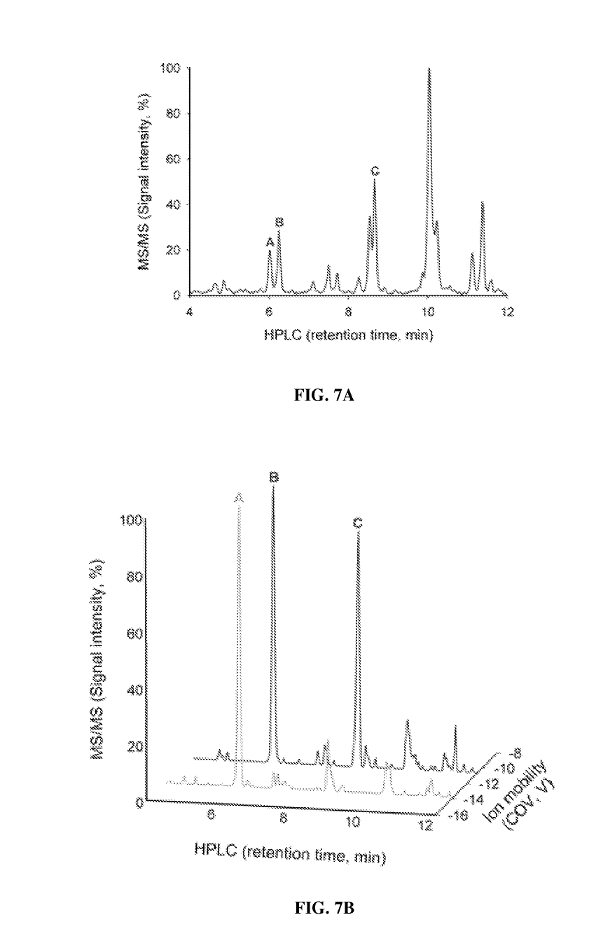

After the chromatographic separation of the isoprostanes as described previously [ref. 8], the samples were introduced in a AB/SCIEX QTRAP 6500 LC/MS/MS System equipped with a SelexION, ion mobility device. The parameters were optimized for each class VI F₂-isoprostanes. A high concentration (3.0%) of 2-propanol was used as the differential mobility spectrometer (DMS) chemical modifier. The other operating parameters were set as follows: DMS temperature=300° C. (high), DMS offset=3.0 V, DMS resolution enhancement=low (22 psi) and separation voltage=3750 V. According to those parameters, the optimal compensation voltage was −13.75 V for iPF_{2α}-VI and −10.62 V for both 5-iPF2α-VI and (±)5-8,12-iso-iPF2α-VI (FIG. 7B). This additional step brings a new dimension of separation to the traditional HPLC-MS/MS (FIG. 7). This technology also improves the limit of detection, enhances the concentration dynamic range and is particularly suitable for lipid isomer separation [ref. 12].

### Example 4

### Relationship with Antioxidant Vitamins

Different ways were evaluated to normalize isoprostanes plasma data according to antioxidant vitamins and the fatty acids content, all factors suspected to influence the level of oxidative stress linked to isoprostanes production in preeclampsia.

In order to do so, protein precipitation was carried out using 2 mL of methanol/ethanol (1/1) containing internal standards (4 ng of β-tocotrienol and 5 ng of ubiquinol-9) on 300 mL of plasma [ref. 9 and 10]. Vitamins were then extracted with 10 mL of hexane using the modified Menke's method [ref. 11]. After centrifugation, the hexane layer was removed, dried under a stream of nitrogen and resuspended in 700 mL of ethanol, then filtered before injection (10 mL) in the HPLC system. The HPLC mobile phase consisted of sonicated methanol/ethanol/isopropanol (88/24/10 v/v/v) containing 15 mM of lithium perchlorate at a flow of 1 mL/min. The column was a Prontosil C18 (4.0 mm×150 mm, 3 mm particle size) preceded by a Prontosil C18 guard cartridge (4.0 mm×10 mm) (Bischoff Chromatography, Atlanta, Ga.). The colometric electrochemical detector (Coulochem III, ESA, Bedford, Mass.) included a guard cell (Model 5020; coulometric electrode at −600 mV) and an analytical cell with two electrodes, the first one adjusted at −150 mV and the second at 600 mV. The current from the second electrode of the analytical cell was electronically recorded and data were integrated using the Beckman gold software (Fullerton, Calif.). Scales were adjusted at 2 mA for vitamin E, 100 nA for ubiquinol-10 and β-carotene, and 50 nA for ubiquinone-10. The amounts of the lipophilic antioxidants were calculated from the ratios of the peak areas of these components to the corresponding internal standard. The β-tocotrienol was used as an internal standard for γ-tocopherol and α-tocopherol. The ubiquinol-9 was used as an internal standard for β-carotene, ubiquinol-10, and ubiquinone-10 [ref. 9].

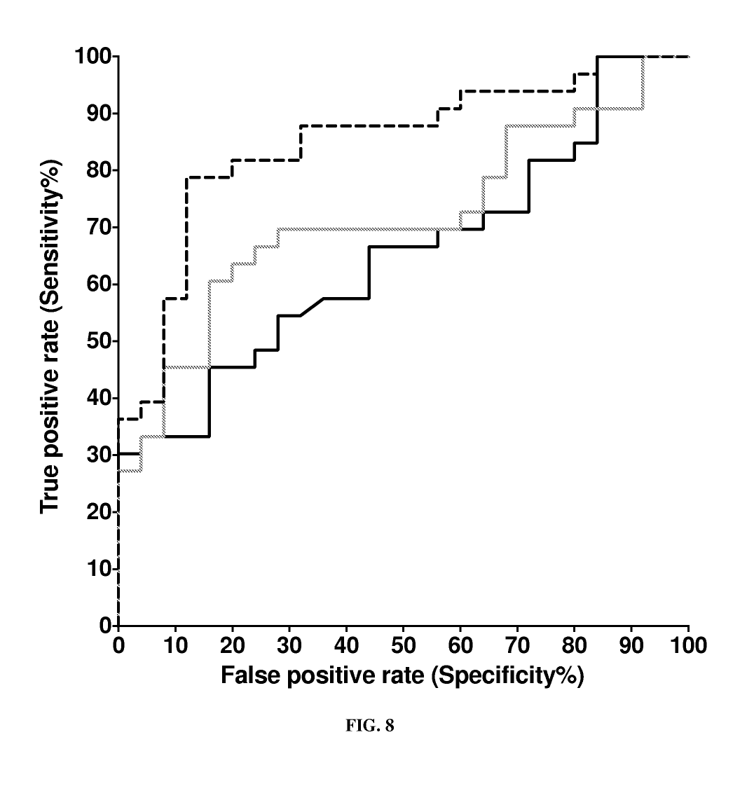

Table 4 shows different ways to normalize isoprostanes plasma data according to antioxidant vitamins and the fatty acids content, all factors suspected to influence the level of oxidative stress linked to isoprostanes production in preeclampsia [ref. 13]. These results comprise a subset of women reported in WO 2013/170369 from which antioxidant plasma vitamins data was available such as β-carotene, vitamin E and coenzyme Q10 (CoQ10) at the first (12-18 weeks) and second (24-26 weeks) routine visit for the pregnancy follow-up. Interestingly, significant differences at the first visit between control and PE were reproduced most of the time at the second visit in Table 4. This makes the test useful throughout pregnancy. Drastic improvement of the preeclampsia (PE) prediction can be observed with β-carotene in ratio with ω-3 fatty acids as normalizing factor (ROC curve area=0.8485 (FIG. 8). Of note, a low β-carotene level during pregnancy is predictive of the later occurrence of PE in our cohort (ROC curve area of 0.8382). The plasma level of β-carotene from 25 normotensive controls (1.07±0.13; mean±SEM) was 2.5-fold-higher than PE pregnancies (0.42±0.08 n=33, P<0.0001 Mann Whitney test) at 12-18 weeks of pregnancies. Indeed, β-carotene levels were also reported lower in severe cases of PE or in PE complicated by diabetes [ref 14]. This aspect is still controversial and may be specific to our cohort though; this is why we propose to control for these levels of β-carotene together with fatty acids.

TABLE 4
Effect of normalization with lipids and antioxidants on the F₂-isoprostanes levels in the first and second trimester
between controls and women that will later develop preeclampsia during the course of their pregnancies.

| | Visit #1 (12-18 weeks)¹ | | | Visit #2 (24-26 weeks)¹ | | |
|---|---|---|---|---|---|---|
| | Normo- | | P² | Normo- | | P² |
| Isoprostanes/ | tensive | Preeclampsia | (Area, ROC | tensive | Preeclampsia | (Area, ROC |
| Normalization factors | (n = 25) | (n = 33) | curve)³ | (n = 23) | (n = 28) | curve)³ |
| (iPF_{2α}-VI + 5 iPF_{2α}-VI) | 154 [124, | 172 [137, 209] | 0.0496* | 155 [135, 173] | 170 [141, 197] | 0.1134 |
| (pg)/ | 175] | | (0.6515) | | | |
| plasma volume (ml) | | | | | | |
| (iPF_{2α}-VI + 5 iPF_{2α}-VI) | 4.4 [1.4, 5.7] | 5.6 [4.3, 7.1] | 0.0069* | 3.2 [2.6, 4.7] | 3.6 [2.9, 4.9] | 0.2153 |
| (pg)/ | | | (0.6915) | | | |
| α-tocopherol (μM) | | | | | | |
| (iPF_{2α}-VI + 5 iPF_{2α}-VI) | 63 [49, 81] | 67 [53, 92] | 0.2720 | 56.1 [37.8, | 60.7 [41.0, | 0.6815 |
| (pg)/ | | | | 85.0] | 103.0] | |
| γ-tocopherol (μM) | | | | | | |
| (iPF_{2α}-VI + 5 iPF_{2α}-VI) | 9.1 [6.1, 16.7] | 13.3 [8.1, 30.7] | 0.1089 | 9.1 [4.0, 12.7] | 10.7 [4.3, 16.4] | 0.4431 |
| (pg)/ | | | | | | |
| α-tocopherol/ | | | | | | |
| γ-tocopherol) | | | | | | |
| (iPF_{2α}-VI + 5 iPF_{2α}-VI) | 4.1 [3.2, 5.1] | 4.8 [4.1, 6.4] | 0.0056* | 2.9 [2.4, 4.4] | 3.4 [2.8, 4.5] | 0.2371 |
| (pg)/ | | | (0.7115) | | | |
| vitamin E (μM)⁴ | | | | | | |
| (iPF_{2α}-VI + 5 iPF_{2α}-VI) | 142 [81, 290] | 645 [340, 1242] | <0.0001** | 197 [124, 434] | 730 [411, | <0.0001** |
| (pg)/ | | | (0.8412) | | 1005] | (0.8447) |
| β-carotene (μM) | | | | | | |
| (iPF_{2α}-VI + 5 iPF_{2α}-VI) | 114 [76, 167] | 149 [115, 215] | 0.0387* | 90.2 [66.7, | 117 [94, 155] | 0.0125* |
| (pg)/ | | | (0.6594) | 114] | | (0.7034) |
| CoQ₁₀ (μM) | | | | | | |
| (iPF_{2α}-VI + 5 iPF_{2α}-VI) | 1058 [665, | 1311 [828, 1712] | 0.0048* | 900 [711, | 1224 [944, | 0.0043* |
| (pg)/ | 1154] | | (0.7152) | 1193] | 1682] | (0.7314) |
| (ω-3/ω-6)^{5, 6} | | | | | | |
| (iPF_{2α}-VI + 5 iPF_{2α}-VI) | 30 [19, 32] | 37 [24, 46] | 0.0030* | 26.7 [20.6, | 34.5 [28.3, | 0.0035 |
| (pg)/ | | | (0.7261) | 31.9] | 48.3] | (0.7360) |
| % ω-3 ⁷ | | | | | | |
| (iPF_{2α}-VI + 5 iPF_{2α}-VI) | 294 [206, | 396 [248, 442] | 0.0059* | 256 [202, 357] | 333 [275, 463] | 0.0089* |
| (pg)/ | 333] | | (0.7103) | | | (0.7127) |
| (PI ω-3/PI ω-6) ^{8, 9} | | | | | | |
| (iPF_{2α}-VI + 5 iPF_{2α}-VI) | 513 [330, | 660 [412, 792] | 0.0048* | 440 [348, 594] | 582 [477, 804] | 0.0070* |
| (pg)/ | 556] | | (0.7152) | | | (0.7189) |
| (UI ω-3/UI ω-6) ^{10, 11} | | | | | | |
| (iPF_{2α}-VI + 5 iPF_{2α}-VI) | 5.2 [3.4, 5.7] | 6.6 [4.4, 8.3] | 0.0032* | 4.8 [3.7, 5.6] | 6.3 [5.0, 8.7] | 0.0035* |
| (pg)/ | | | (0.7248) | | | (0.7360) |
| UI ω-3 | | | | | | |
| (iPF_{2α}-VI + 5 iPF_{2α}-VI) | 3.9 [2.7, 4.5] | 5.1 [3.5, 6.5] | 0.0039* | 3.7 [2.8, 4.4] | 5.0 [3.9, 6.8] | 0.0035* |
| (pg)/ | | | (0.7200) | | | (0.7360) |
| PI ω-3 | | | | | | |
| (iPF_{2α}-VI + 5 iPF_{2α}-VI) | 24 [14, 35] | 7.8 [5, 22] | 0.0003** | 19.7 [11.7, | 7.2 [5.3, 14.1] | 0.0002** |
| (pg)/ | | | (0.7721) | 29.1] | | (0.7950) |
| (% ω-3/β-carotene) | | | | | | |
| (iPF_{2α}-VI + 5 iPF_{2α}-VI) | 30 [15, 48] | 7.8 [3.9, 13] | <0.0001** | 27.3 [13.7, | 6.3 [4.2, 9.0] | <0.0001** |
| (pg)/ | | | (0.8485) | 31.7] | | (0.8494) |
| (UI ω-3/β-carotene) | | | | | | |
| (iPF_{2α}-VI + 5 iPF_{2α}-VI) | 3.5 [1.9, 5.0] | 1.1 [0.7, 3.0] | 0.0004** | 2.8 [1.6, 4.1] | 1.0 [0.7, 2.0] | 0.0002** |
| (pg)/ | | | (0.7633) | | | |
| (PI ω-3/β-carotene) | | | | | | |
| (iPF_{2α}-VI + 5 iPF_{2α}-VI) | 6.0 [3.6, 12.4] | 26 [11.5, 59] | <0.0001** | 9.8 [5.9, 18.3] | 39.36 [22.7, | <0.0001** |
| (pg)/ | | | (0.8194) | | 60.8] | (0.8294) |
| (UI ω-3/ | | | | | | |
| (β-carotene/CoQ10)) | | | | | | |
| (iPF_{2α}-VI + 5 iPF_{2α}-VI) | 114 [52, 216] | 45 [24, 77] | 0.0011** | 88 [59, 145] | 43 [25, 72] | <0.0024** |
| (pg)/ | | | (0.7467) | | | (0.7453) |
| (UI ω-3/(β-carotene/ | | | | | | |
| (CoQ10/α-tocopherol))) | | | | | | |

¹Values are medians and quartiles [Q1, Q3];
²Mann-Whitney test;
³ROC = receiver operating characteristic;
⁴Vitamin E = α-tocopherol + γ-tocopherol;
⁵Omega-3 fatty acids = C18:3ω3 + C18:4ω3 + C20:3ω3 + C20:4ω3 + C20:5ω3 + C22:5ω3 + C22:6ω3
⁶Omega-6 fatty acids = C18:1ω6 + C18:2ω6 + C18:3ω6 + C20:2ω6 + C20:3ω6 + C20:4ω6 + C22:2ω6 + C22:4ω6 + C22:5ω6
⁷ ω-3 % = all ω-3 described in [^{5.}]/total of all fatty acids described × 100 in ref 15.
⁸ Peroxidation index for omega-3 (PI ω-3) = (% Trienoic × 2) + (% Tetraenoic × 4) + (% Pentaenoic × 6) + (% Hexaenoic × 8) of fatty acids described above in [^{5.}]
⁹ Peroxidation index for omega-3 (PI ω-6) = (% Monoenoic × 0.025) + (% Dienoic × 1) + (% Trienoic × 2) + (% Tetraenoic × 4) + (% Pentaenoic × 6) of fatty acids described above in [^{6.}]
¹⁰ Unsaturation index for omega-3 (UI ω-3) = (% Monoenoic × 1) + (% Dienoic × 2) + (% Trienoic × 3) + (%Tetraenoic × 4) + (% Pentaenoic × 5) + (% Hexaenoic × 6) of fatty acids described above in [^{5.}]
¹¹ Unsaturation index for omega-6 (UI ω-6 ) = (% Monoenoic × 1) + (% Dienoic × 2) + (% Trienoic × 3) + (% Tetraenoic × 4) + (% Pentaenoic × 5) + (% Hexaenoic × 6) of fatty acids described above in [^{6.}]

### Example 5

### Conclusion

In conclusion, the data reports gestational trends in the total fatty acid profile associated with PE. Our investigation has shown early and selective changes in markers of oxidative stress, fatty acids and mostly the total fatty acid profile suggesting that these may play a role in the aetiology of the disease. Especially significant is the measure of 5-iPF_{2α}-VI and/or iPF_{2α}-VI when assessed as the ratio against: a) the ratio of omega-3 PUFA over omega-6 PUFA; b) the percentage of omega-3 PUFA; or c) the ratio of omega-3 PUFA unsaturation index over β-carotene; d) the ratio of omega-3 PUFA unsaturation index over β-carotene over CoQ10; or e) the ratio of omega-3 PUFA unsaturation index over β-carotene over CoQ10 over α-tocopherol. Since abnormal profiles were demonstrated several weeks before the clinical onset of PE, we were able to identify combinations of markers that have the potential to identify women who will later develop PE.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

All patents, patent applications and publications mentioned in this specification are herein incorporated by reference to the same extent as if each independent patent, patent application, or publication was specifically and individually indicated to be incorporated by reference.

### REFERENCES

1. Chappell et al., The Lancet, 354, 810-816, 1999.
2. Xu et al., An International trial of Antioxydants in the Prevention of Preeclampsia (INTAPP), Am. J. Ostetric & Gynecol. 239.e1, 2010)
3. Counil, E., et al., Association between trans-fatty acids in erythrocytes and pro-atherogenic lipid profiles among Canadian Inuit of Nunavik: possible influences of sex and age. Br J Nutr, 2009. 102(5): p. 766-76.
4. Taylor, A. W., et al., Benefits of prolonged gradient separation for high-performance liquid chromatography-tandem mass spectrometry quantitation of plasma total 15-series F-isoprostanes. Anal Biochem, 2006. 350(1): p. 41-51.
5. Roland, L., et al., Existence of compensatory defense mechanisms against oxidative stress and hypertension in preeclampsia. Hypertens Pregnancy, 2010. 29(1): p. 21-37.
6. Nigam, A.; Frasure-Smith, N.; Lesperance, F.; Julien, P. Relationship between n-3 and n-6 plasma fatty acid levels and insulin resistance in coronary patients with and without metabolic syndrome. Nutrition, metabolism, and cardiovascular diseases 19:264-270; 2009.
7. Counil, E.; Julien, P.; Lamarche, B.; Chateau-Degat, M. L.; Ferland, A.; Dewailly, E. Association between trans-fatty acids in erythrocytes and pro-atherogenic lipid profiles among Canadian Inuit of Nunavik: possible influences of sex and age. Br J Nutr 102:766-776; 2009.
8. Larose, J., P. Julien, and J. F. Bilodeau. 2013. Analysis of F2-isoprostanes in plasma of pregnant women by HPLC-MS/MS using a column packed with core-shell particles. J Lipid Res 54: 1505-1511.
9. Finckh B, Kontush A, Commentz J, Hubner C, Burdelski M, Kohlschutter A. (1995) Monitoring of ubiquinol-10, ubiquinone-10, carotenoids, and tocopherols in neonatal plasma microsamples using high-performance liquid chromatography with coulometric electrochemical detection. Anal Biochem 232:210-216.
10. Lang J K, Gohil K, Packer L. (1986) Simultaneous determination of tocopherols, ubiquinols, and ubiquinones in blood, plasma, tissue homogenates, and subcellular fractions. Anal Biochem 157:106-116.
11. Menke T, Niklowitz P, Adam S, Weber M, Schluter B, Andler W (2000) Simultaneous detection of ubiquinol-10, ubiquinone-10, and tocopherols in human plasma microsamples and macrosamples as a marker of oxidative damage in neonates and infants. Anal Biochem 282:209-217.
12. Kliman, M., May, J. C., and McLean, J. A. (2011) Lipid analysis and lipidomics by structurally selective ion mobility-mass spectrometry. Biochimica et biophysica acta 1811, 935-945
13. Bilodeau, J. F. (2014) Review: maternal and placental antioxidant response to preeclampsia—impact on vasoactive eicosanoids. Placenta 35 Suppl, S32-38
14. Azar, M., Basu, A., Jenkins, A. J., Nankervis, A. J., Hanssen, K. F., Scholz, H., Henriksen, T., Garg, S. K., Hammad, S. M., Scardo, J. A., Aston, C. E., and Lyons, T. J. (2011) Serum carotenoids and fat-soluble vitamins in women with type 1 diabetes and preeclampsia: a longitudinal study. Diabetes care 34, 1258-1264.
15. Larose, J., Julien, P., Greffard, K., Fraser, W. D., Audibert, F., Wei, S. Q., and Bilodeau, J. F. (2014) F-isoprostanes are correlated with trans fatty acids in the plasma of pregnant women. Prostaglandins, leukotrienes, and essential fatty acids in Press.

1. A method for measuring blood isoprostane profile in a pregnant woman at risk of developing preeclampsia (PE), comprising the steps of:
a) extracting lipids from a said pregnant woman's biological sample;
b) measuring total level of F₂-isoprostane class VI;
c) optionally, measuring total level of 15(R)-PGF_{2α} or fatty acids from said sample;
d) optionally, establishing a ratio of F₂-isoprostane class VI over blood fatty acids for said pregnant woman;
e) comparing said amount or said ratio with a control level or ratio from a control population or individual representative of said pregnant woman;
f) reporting said comparison from step e) to said subject's treating physician;
wherein when said level or ratio is at least about 10% higher than said control level or ratio, said physician may diagnose pre-eclampsia and, optionally take measures to monitor or treat said pregnant woman.

2. The method according to claim 1, further comprising before step a), a step of separating isoprostanes from said extracted lipids, wherein said separating is carried out by mass spectrometry.

3. The method according to claim 2, wherein said F₂-isoprostane class VI is selected from the group consisting of: 5-iPF_{2α}-VI and iPF_{2α}-VI.

4. The method according to claim 3, wherein said fatty acid is selected from the group consisting of: arachidonic acid, omega-3 and omega-6 polyunsaturated fatty acids (PUFA).

5. The method according to claim 4, wherein said ratio is a ratio of (±)iPF_{2α}-VI isoprostane over the ratio of omega-3 to omega-6 polyunsaturated fatty acids (PUFA), whereby when said ratio is above a control ratio is indicative that said pregnant woman is at risk of developing PE.

6. The method according to claim 1, wherein said ratio is a ratio of (±)iPF_{2α}-VI isoprostane over percentage of omega-3 polyunsaturated fatty acids (PUFA) in said sample, whereby when said ratio is above a control ratio is indicative that said pregnant woman is at risk of developing PE.

7. The method according to claim 1, wherein said ratio is a ratio of (±)iPF_{2α}-VI isoprostane over a ratio of omega-3 PUFA unsaturation index over concentration of β-carotene, whereby when said ratio is above a control ratio is indicative that said pregnant woman is at risk of developing PE.

8. The method according to claim 1, wherein said ratio is a ratio of (±)iPF_{2α}-VI over a ratio of omega-3 PUFA unsaturation index over concentration of β-carotene over CoQ10, whereby when said ratio is above a control ratio is indicative that said pregnant woman is at risk of developing PE.

9. The method according to claim 1, wherein said ratio is a ratio of (±)iPF_{2α}-VI over a ratio of omega-3 PUFA unsaturation index over concentration of β-carotene over CoQ10 over α-tocopherol, whereby when said ratio is above a control ratio is indicative that said pregnant woman is at risk of developing PE.

10. The method according to claim 1, wherein said biological sample is selected from the group consisting of: blood, plasma, serum and blood cell membranes.

11. The method according to claim 1, wherein said control population or individual is selected from the group consisting of: an individual in a normal population devoid of PE symptom; a non-pregnant woman; same pregnant subject prior to pregnancy; and same pregnant subject prior to 10 week of pregnancy.

12. The method according to claim 1, wherein said amount or said ratio is increased by at least about 15%.

13. An assay for predicting the appearance of preeclampsia (PE) in a pregnant woman comprising the steps of:
a) obtaining a sample from said pregnant woman;
b) assessing amount of total isoprostane in said sample;
c) assessing blood fatty acid profile in said sample;
d) establishing a ratio of total isoprostanes over blood fatty acid profile for said woman;
e) comparing said ratio with a control level for a population or an individual representative of said pregnant woman; and
f) determining if said comparing of step e) is higher than about 10% of said control level; and
g) optionally reporting said determination from step f) to said subject's treating physician.

14. The assay according to claim 13, wherein said control level is established with a control population or individual, wherein said control population or individual is selected from the group consisting of: an individual in a normal population devoid of PE symptom; a non-pregnant woman; same pregnant subject prior to pregnancy; and same pregnant subject prior to 10 week of pregnancy.

15. The assay according to claim 13, wherein said amount or said ratio is increased by at least about 15%.

16. The assay according to claim 13, wherein said ratio is a ratio of isoprostane over the ratio of omega-3 to omega-6 polyunsaturated fatty acids (PUFA), wherein when said ratio is above a control ratio is indicative that said pregnant woman is at risk of developing PE.

17. The assay according to claim 13, wherein said ratio is a ratio of isoprostane over percentage of omega-3 polyunsaturated fatty acids (PUFA), wherein when said ratio is above a control ratio is indicative that said pregnant woman is at risk of developing PE.

18. The assay according to claim 13, wherein said ratio is a ratio of isoprostane over a ratio of omega-3 PUFA unsaturation index over concentration of β-carotene, wherein when said ratio is above a control ratio is indicative that said pregnant woman is at risk of developing PE.

19. The assay according to claim 13, wherein said amount or said ratio is increased by at least about 15%.

20. The assay according to claim 13, wherein said biological sample selected from the group consisting of: blood, plasma, serum and blood cell membranes.
